(19) United States
(12) Patent Application Publication
(10) Pub. No.: US 2025/0134448 A1
(43) Pub. Date: May 1, 2025

(54) Systems And Methods For Seizure Forecasting Using Self-Use, Non-Invasive Technologies

(71) Applicants: Shriya Jaddu, Great Falls, VA (US); Sidh Jaddu, Great Falls, VA (US)

(72) Inventors: Shriya Jaddu, Great Falls, VA (US); Sidh Jaddu, Great Falls, VA (US)

(21) Appl. No.: 18/929,365

(22) Filed: Oct. 28, 2024

Related U.S. Application Data
(60) Provisional application No. 63/546,218, filed on Oct. 29, 2023.

(51) Int. Cl. A61B 5/00; G16H 40/67; G16H 50/30

(52) U.S. Cl. CPC A61B 5/4094; A61B 5/681; A61B 5/7267; A61B 5/7275; G16H 40/67; G16H 50/30

(57) ABSTRACT

A system for seizure forecasting is provided. The system comprises a wearable device, and a mobile device. The wearable device includes one or more sensors that are configured to obtain physiological data of a user that describes pre-ictal, ictal and post-ictal and normal phases of the user's seizure related activity. The mobile device includes at least a processor, one or more machine learning models and a memory. The memory is encoded with instructions that, when executed by the at least processor of the system, cause the system to perform operations comprises periodically receiving the physiological data of the user from the wearable device; and analyzing the received physiological data using the machine learning models and forecast upcoming seizures.

### BACKGROUND

### Field

The present patent application relates to systems and methods for seizure forecasting and alerting using self-use, non-invasive technologies.

### Description of Related Art

Characterized by episodes of abnormal neuron activity, epilepsy is a neurological disorder that affects more than seventy million people worldwide. Epilepsy is a diverse neurological disorder, and not all epileptic patients have the same symptoms both before seizures (pre-ictal period), during seizures (ictal period), and after seizure (post-ictal period).

Although there are more than 40 drug treatments available to prevent seizures, a third of epilepsy patients around the world (around 23 million) suffer from drug-resistant epilepsy (DRE), causing them to have uncontrolled seizures even when on seizure-prevention medications. During seizure events, patients may zone out, fall unconscious, lose control of parts of their body, and may have bodily injuries. They also experience a period of disorientation after their seizures which can last minutes to hours. Because of these symptoms as well as the randomness of seizures, DRE patients lead restricted lifestyles due to safety and social issues. For example, in many states and countries, people with epilepsy are not allowed to drive. DRE patients are often hesitant to perform activities such as swimming, hiking, etc. with the worry that they may drown or they may have bodily harm and other injuries. Similarly, they are also hesitant to involve themselves in social activities, as they are worried about stigma and embarrassment. Therefore, predicting and alerting upcoming seizure activity in epileptic patients is incredibly important.

Currently, there are three approaches to DRE: surgical procedures, detection systems, and prediction systems. For focal seizures that originate in a specific part of the brain, surgery to remove the part of the brain causing seizures is a viable procedure for some patients to stop seizures. Other surgical procedures include stimulations such as Deep Brain Stimulation, Vagal Nerve Stimulation, etc. In Deep Brain Stimulation, mild electrical currents are delivered to the brain to stimulate it and prevent seizures. In Vagus Nerve Stimulation, stimulators generate pulses that create low-energy electrical signals, and leads carry these pulses to the vagus nerve. These solutions are costly, unreliable, and inaccessible to most DRE patients (especially those who reside in developing countries and have limited diagnosis and treatment access). In addition, more than half of epilepsy patients who have surgery have seizure recurrence, and stimulation devices rarely stop seizures completely.

Several researchers and companies, including Empatica and the Epilepsy Foundation, have been utilizing physiological and EEG data to detect seizures and alert caregivers. These devices are effective at only detecting seizures during the ictal period (while seizures are occurring); however, they do not properly address the social and safety issues associated with DRE, given that they do not predict upcoming seizures and that the patients are unable to take precautions to prevent harm, social embarrassment, or unease. Additionally, a majority of these devices can only detect convulsive seizures during the ictal period (e.g., generalized tonic-clonic seizures).

Finally, for pre-ictal seizure prediction, researchers have mainly been exploring the use of Electroencephalography (EEG) since EEG data shows abnormal brain activity during the pre-ictal period. There are two well-known methods of EEG collection for seizure prediction: subcutaneous EEG and ear-EEG devices. Subcutaneous EEG is an invasive method in which electrodes are implanted into the brain to record electrical activity and make predictions based on changes in electrical activity. Similar to the surgical seizure-stopping procedures mentioned above, subcutaneous EEG-based prediction is expensive and invasive, making it impractical for most patients. Ear-EEG, an EEG collection device placed over/on the ear, is a non-invasive alternative to subcutaneous EEG implants. However, this approach is impractical for 24-hour use by patients, and, in past studies using ear-EEG devices, patients reported discomfort and discontinued use.

The most effective solution/approach to this problem is seizure prediction (during the pre-ictal period) using non-invasive physiological and physical data. Research has shown that patients can experience changes in physiological features such as blood volume pulse, electrodermal activity, heart rate, etc. during the pre-ictal period. Additionally, patients have reported changes in physical features such as slurred speech. Although few researchers have attempted to use physiological data to predict seizures during the pre-ictal period, they did not have acceptable performances for practical use.

### BRIEF DESCRIPTION OF THE DRAWINGS

Various embodiments are disclosed, by way of example only, with reference to the accompanying schematic drawings in which corresponding reference symbols indicate corresponding parts, in which

FIG. 1 shows an exemplary system for seizure forecasting and alerting according to an embodiment of the present patent application;

FIG. 2 shows an exemplary system for seizure forecasting and alerting according to another embodiment of the present patent application;

FIG. 3 shows an exemplary wearable device of the system for seizure forecasting and alerting according to an embodiment of the present patent application;

FIG. 4 shows an exemplary mobile device of the system for seizure forecasting and alerting according to an embodiment of the present patent application;

FIG. 5 shows an exemplary cloud device/server of the system for seizure forecasting and alerting according to an embodiment of the present patent application;

FIG. 6 shows an exemplary system architecture of the system for seizure forecasting and alerting according to an embodiment of the present patent application;

FIG. 7 shows an exemplary format of a single training sample according to an embodiment of the present patent application;

FIG. 8 shows an exemplary image showing the system displaying the seizure alert on the mobile application according to an embodiment of the present patent application; and

FIG. 9 shows an exemplary image of the personalization features in the mobile application.

### DETAILED DESCRIPTION OF THE DRAWINGS

This patent application provides an effective end-to-end seizure prediction system using physiological data (e.g., heart rate, blood volume pulse, electrodermal activity, temperature, accelerometry, etc.) and physical data (e.g., voice/audio). This patent application also provides a novel deep learning-enabled ecosystem that utilizes transfer-learning-like methods to personalize and optimize predictions for individual DRE patients. Additionally, the ecosystem may be configured to voice/audio tests as a “second opinion” for the predictions made using physiological data.

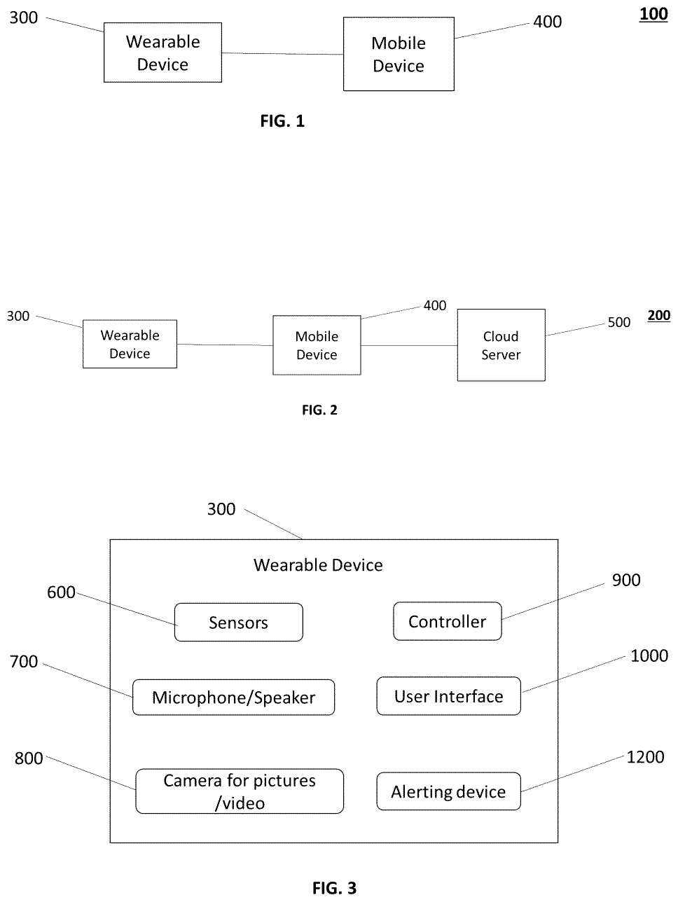

FIG. 1 shows an exemplary system 100 for seizure forecasting and alerting according to an embodiment of the present patent application. The system 100 may include a wearable device 300 and a mobile device 400. FIG. 2 shows an exemplary system 200 for seizure forecasting and alerting according to another embodiment of the present patent application. The system 200 may include the wearable device 300, the mobile device 400, and a cloud device/server 500.

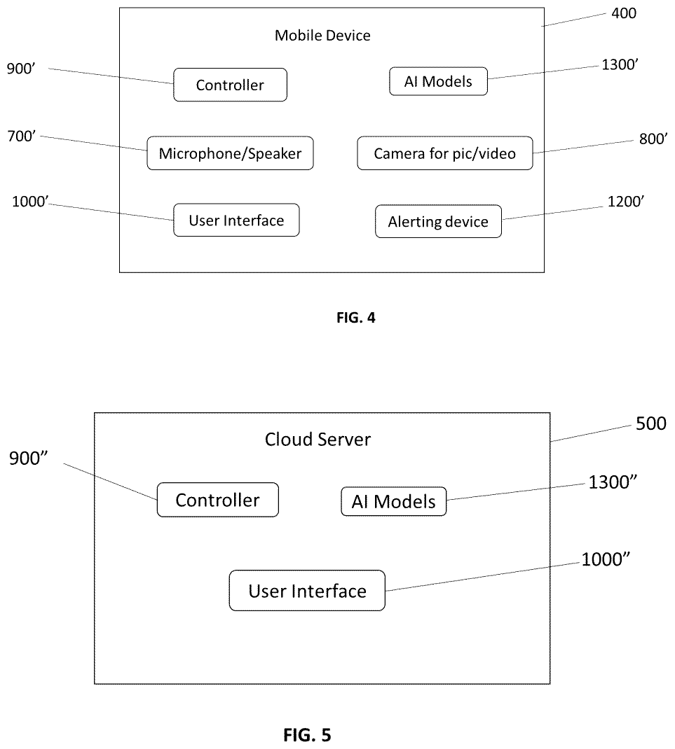

The exemplary wearable device 300 is shown in FIG. 3. The wearable device 300 may include, but not limited to, one or more sensors 600, a microphone and a speaker 700, a camera 800 for pictures/video, a controller 900, a user interface 1000 and an alerting device 1200. The exemplary mobile device 400 is shown in FIG. 4. The mobile device 400 may include, but not limited to, a microphone and a speaker 700′, a camera 800′ for pictures/video, a controller 900′, a user interface 1000′, an alerting device 1200′ and one or more AI models 1300′. The exemplary cloud server 500 is shown in FIG. 5. The cloud server 500 may include, but not limited to, a controller 900″, a user interface 1000″, and one or more AI models 1300″. The components of the wearable device 300, the mobile device 400 and the cloud server 500 are all described in more detail in discussions below. Each of the controller 900 of the wearable device 300, the controller 900′ of the mobile device 400, and the controller 900″ of the cloud server 500 has one or more processors.

The components of the wearable device 300 are all in communication with each other. For example, the one or more sensors 600, the microphone and the speaker 700, and the camera 800 may be configured to transmit data to the controller 900. The one or more sensors 600, the microphone and the speaker 700, and the camera 800 may also be configured to transmit data to the mobile device 400 and/or the cloud server 500. The controller 900 may be configured to process the received data and transmit the processed data to the user interface 1000 and/or the alerting device 1200. The controller 900 may also be configured to transmit received data to the mobile device 400 and/or the cloud server 500.

The components of the mobile device 400 are all in communication with each other. The controller 900′, the user interface 1000′, and the one or more AI models 1300′ of the mobile device 400 may form a mobile application on the mobile device 400. The microphone and the speaker 700′ and the camera 800′ may be configured to transmit data to the controller 900′ and/or the AI models 1300′. The microphone and the speaker 700′ and the camera 800′ may also be configured to transmit data to the cloud server 500. The controller 900′ and/or the AI models 1300′ may be configured to process the received data and transmit the processed data to the user interface 1000′ and/or the alerting device 1200′. The controller 900′ and/or the AI models 1300′ may also be configured to transmit received data to the cloud server 500. The controller 900′ and/or the AI models 1300′ may also be configured to receive the data and run some algorithmic logic related to seizure forecasting.

The components of the cloud server 500 are all in communication with each other. The controller 900″, the user interface 1000″, and the one or more AI models 1300″ of the cloud server 500 may form a cloud application on the cloud server 500. The controller 900″ and/or the AI models 1300″ may be configured to receive data from the wearable device 300 and/or the mobile device 400 and may be configured to process data and transmit processed data to the wearable device 300 and/or the mobile device 400. The controller 900″ and/or the AI models 1300″ may also be configured to receive the data and run some algorithmic logic related to seizure forecasting.

The use of the same or similar reference numerals in different figures indicates similar, related, or identical items. The embodiments have many identical parts in function, and therefore the description may have been omitted. Where the operation of such embodiment features/parts is not described, the reader may assume that the operations are the same as described in other portions of the present patent application, having regard to those modifications apparent to a person of ordinary skill in the art.

The wearable device 300 may be worn by a patient/user. A patient or user may refer to a person who may be epileptic and who may be interacting with the wearable device 300 (e.g., wearing it) and/or interacting with the mobile device 400. The wearable device 300 may include a wrist/arm/hand/leg worn device, a head worn device, a body/chest worn device, etc. The wearable device 300 may include one or more sensors 600 that are each configured to measure (e.g., non-invasively) and transmit physiological parameters of a patient/user. The wearable device 300 may be configured to be convenient, comfortable, and portable for 24-hour use. The wearable device 300 including the sensor(s) 600 may be configured to be in communication with a controller 900 having one or more processors. The controller 900 may be configured to receive the sensor data associated with a user. The controller 900 may be configured to receive the sensor data associated with a user in real-time. The controller 900 may be configured to receive the sensor data associated with a user continuously or periodically. The controller 900 may be configured to receive the sensor data associated with a user intermittently. The wearable device 300 may use a wireless (e.g., Bluetooth, WIFI, Zigbee) transceiver and connection to transmit the sensor data to a mobile application that runs on the user's smartphone, tablet, or other mobile or personal device. An electronic device or mobile communications device (e.g., a smartphone) may execute a mobile application that includes executable program code that directs the smartphone (or mobile device 400) to communicate with the wearable device 300, a server and/or a cloud/remote system/server 500.

The data received may include: i) electrodermal activity data, ii) heart rate data, iii) blood volume pulse data, iv) temperature data, v) accelerometer-based movement data, vi) time data, vii) date data, viii) global positioning system data, ix) user confirmed seizures/seizure data, x) self-reported seizures/seizure data, or xi) combinations thereof. The wearable device 300 may also be configured to provide other data including i) time data, ii) date data, iii) global positioning system data, iv) self-reported seizure data, or v) combinations thereof.

The wearable device 300 may include sensors 600 that continuously capture patient information including blood volume pulse (BVP), electrodermal activity (EDA), heart rate (HR), accelerometry, and temperature. The wearable device 300 may be configured to send the patient information to the mobile application on the patient's or caregiver's phone with WiFi or without WiFi (e.g., using Bluetooth).

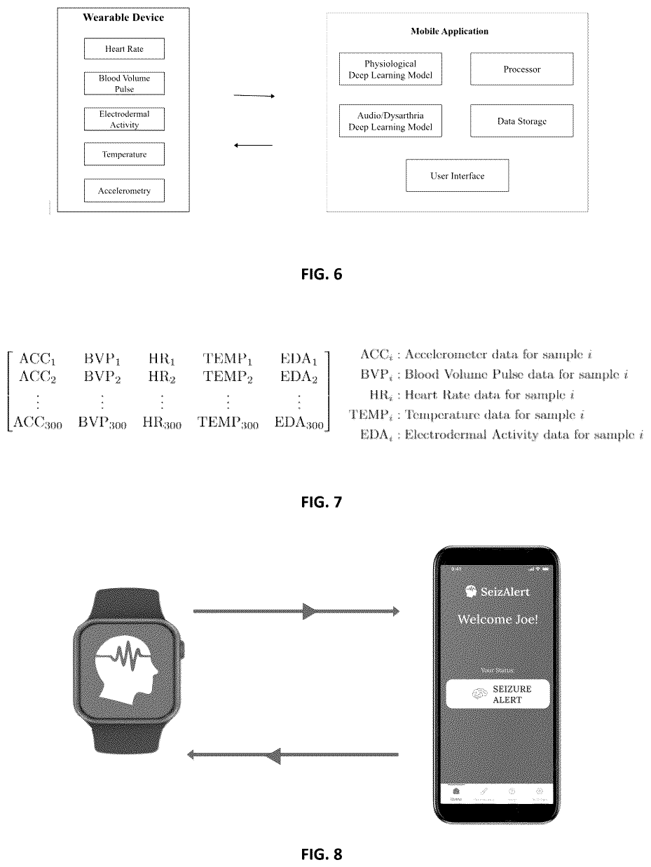

As shown in FIG. 6, the system may include a mobile application and the wearable device. The wearable device 300 may include five sensors 600 that are configured to collect physiological data from the patient as shown in FIG. 6. The sensors 600 may include an electrodermal activity sensor that is configured to measure the electrodermal activity of the patient/user; a heart rate sensor that is configured to measure the heart rate of the patient; a blood volume pulse sensor that is configured to measure the blood volume pulse of the patient; a temperature sensor that is configured to measure the temperature of the patient; an accelerometer that is configured to measure accelerometer-based movement data of the patient, and a global positioning system configured to measure actigraphy data (e.g., accelerometer-based and location-based activity data), etc. The term “electrodermal activity” refers to a measure of neurally mediated effects on sweat gland permeability, observed as changes in the resistance of the skin to a small electrical current, or as differences in the electrical potential between different parts of the skin. In one embodiment, the number of sensors in the wearable device 300 may vary.

As used herein, the term “ictal” refers to the period a physiologic state/event such as a seizure, and may be used to further indicate the period of a, e.g., stroke, headache, inflammation, flare-up, mental health episode, or in general any relapsing-remitting diseases; the term “preictal” or “pre-ictal” may refer to the time period preceding (i.e., before) an ictal event of variable duration; the term “postictal” may refer to the period refers to the state shortly after an ictal event; the term “interictal” or “normal” may refer to the period between post-ictal event and the next pre-ictal event.

The mobile application may be installed/deployed on the patient's or caregiver's phone. The mobile application may run on the user's smartphone, tablet, or other mobile or personal device. The basic elements/components of the mobile application are shown in FIG. 6. The mobile application may include other elements/components, as would be appreciated by a person skilled in the art, that are not shown in FIG. 6. As shown in FIG. 6, the mobile application may be configured and designed to host/include multiple deep learning models along with other components. FIG. 6 presents an example configuration with a physiological model, and audio/dysarthria model. The deep learning models may be deployed on the mobile application as TensorFlow Lite models or technology models, so predictions as well as the training of these models could be performed on the phone/mobile application. The deep learning models may interchangeably referred to as AI models 1300, 1300′, or 1300″.

The physiological/primary model may be a deep learning model that is trained on/with physiological data from epileptic patients so it could predict upcoming seizures based on the patient's physiological data from the wearable device 300. The physiological model may also be configured to be further trained (personalized) on the mobile device/application using the data received from the wearable device 300 to improve the model accuracies for that patient. The physiological model may be used for binary classification of pre-ictal and interictal (normal) data and may be configured to predict upcoming seizures. The physiological model may be deployed on the user's or caregiver's phone 400 on which the mobile application is installed. The methodology of the data preprocessing and model building for the physiological model are explained in detail below.

The data used for modeling the physiological model may include blood volume pulse, electrodermal activity, heart rate, accelerometry, and temperature information. Blood volume pulse may refer to the rate of blood flow and may be measured as the heart beats. Electrodermal activity may refer to the electrical activity of the skin but also may include characteristics such as sweating. Accelerometry may be a measure of movement. The accelerometry data may include data from three channels (X, Y, Z) as well the magnitude of these channel values. As part of pre-processing, segments of pre-ictal and interictal data for all epileptic patients may be selected for training and testing from the data. The selected data may include data from seizures of various types and symptoms including generalized tonic-clonic, clonic, focal (motor, complex, and discognitive), myoclonic, hypermotor, and subclinical. Relatively equal amounts of interictal and pre-ictal data may be selected to prevent major class imbalances in subsequent modeling.

The physiological data may be scaled using Robust Data Scaling. The scaled values may be calculated using Equation 1 as shown below. In Equation 1, X represents the original value, X_{median} represents the median of all of the data points, X_{new} represents the scaled value, and IQR is the interquartile range.

$${X}_{new} = \frac{X - {X}_{median}}{IQR} \qquad Equation 1$$

The median value of all of the data points may be calculated and subtracted from the original data value, and the resulting value may be divided by the interquartile range, which is defined as the range between the 25% and 75% percentiles of the data. Robust scaling may not only reduce the impact of outliers but also may help with the modeling process (e.g., if all of the data points were originally extremely large numbers).

As the deep learning models may use segments of pre-ictal and interictal (normal) data for training and testing, the data may be broken down into arrays of 30 or 60 or other seconds of data for the time-series classification model. A sample duration of 30 seconds may be chosen to ensure that training samples were long enough for models to accurately analyze and understand patterns in the data. The exemplary format of a single training sample is shown in FIG. 7. In one embodiment, three separate datasets may be randomly created for modeling: a training dataset (60% of the original data), a validation dataset (20% of the original data), and a testing dataset (20% of the original data).

In one embodiment, several multivariate model architectures including BiLSTM, CNN-LSTM, and CNN-BiLSTM were experimented with. A multivariate model may be a model trained with data comprising multiple variables/features. In this data, the multiple variables may represent the data from the five sensors. A Convolutional Neural Network-based Bidirectional Long Short Term Memory Network (CNN-BiLSTM) architecture showed better performance when compared with other model architectures. A Convolutional Neural Network is a deep-learning model optimized for learning spatial relationships. CNNs are also very effective at discovering relationships between features in multivariate models, given their ability to find spatial patterns. Bidirectional LSTM networks are a type of recurrent neural network that is used for time series analysis and natural language processing. Specifically, the bidirectional element of the model architecture allows it to see patterns both in the previous and upcoming data, making it ideal for understanding patterns in time sequence seizure data with more subtle changes. The system of this patent application may include a hybrid of these two architectures CNN-BiLSTM, allowing the model to effectively observe and learn both temporal and spatial patterns in the seizure data. Although the multivariate CNN-BiLSTM models performed very well, different single variate models were also trained with data from different sensors to understand whether a multivariate model approach or single variate model approach would perform better. The multivariate model outperformed all the single variate models.

The selected CNN-BiLSTM multivariate model architecture began with 3 1D Convolutional layers, with 512, 256, and 128 units, respectively. Each of the layers may use a Rectified Linear Unit (ReLU) activation function. Following the three Convolutional layers, 1D Max Pooling may be employed to extract the most significant features. Next, the model may include a Bidirectional LSTM layer with 128 units. As mentioned above, bidirectional layers perform very well in sequence classification tasks, as they consider temporal patterns in both the forward and backward directions. The output of this layer may be flattened into a 1D vector. Finally, two dense layers may be implemented. The first dense layer may include 64 units and may use ReLU activation and the second dense layer (output layer) may include 1 unit and may use sigmoid activation, considering that sigmoid activation is suitable for binary classification tasks.

All modeling of this patent application may be performed using the Keras TensorFlow library. Hyperparameters may be selected after extensive tuning. The model may be trained on the training dataset for 10 epochs. A batch size of 32 may be selected for the model. The model may be validated using the validation set and may be evaluated using the binary cross-entropy loss function. The model may use the Adam optimizer with a learning rate of 0.001. A Keras callback called ReduceLRonPlateau may be implemented to reduce the learning rate if no improvement was seen in the model after 3 epochs.

Additionally, class weights may be implemented to correct the imbalance using Equation 2 (as shown below), if the training data used has any imbalance between pre-ictal data and interictal data:

$$Class Weight = \frac{total samples}{number of classes \times class count} \qquad Equation 2$$

In Equation 2 above, the total sample number may be defined as the number of samples in the training dataset, the number of classes may be two (considering that the model was carrying out binary classification), and the class count may be the number of samples per class in the training dataset.

The model may be trained and tested with the data from multiple epileptic patients. This model may be considered the “general” model, as it may be trained on a diverse set of epilepsy patient data. To confirm the efficacy of the general model for each of the patients, the general model may be tested on data of each of the individual patients. Although the general model was able to predict relatively well about half of the patients, the model did not predict as well for some of the other patients. As mentioned in the introduction, patients may have different pre-ictal patterns, making it challenging for a general model to effectively predict seizures for all seizure patients.

To combat this, this application implemented personalization/individualization. In this, the general model may be used as a base, and specific patient data may be used to train on top of the existing general model. The patient-specific data samples may be given about twice as much weight as the base data, to ensure that the model was maintaining past learnings from the general pool of data, while also optimizing to fit the needs of the patient. To test the effectiveness of this personalization technique for a patient, the patient was removed from the training set, the “general” model was trained and tested on the remaining patients' data, and the model was further trained and tested on the patient's specific data.

The dysarthria/audio model may be trained with audio/dysarthria recordings, so it could provide a “second opinion” based on the patient's voice clip. That is, an additional model may be built to identify patients' physical pre-ictal symptoms (e.g., changes in vocal tone). As aforementioned, studies have shown that epileptic patients may experience vocal changes such as slurred speech (dysarthria) during the pre-ictal period. Thus, a second prediction system may be created to provide an optional “second opinion” to the alerts generated using the physiological model. The audio/dysarthria model may be deployed on the mobile application. The second system may be implemented such that it would only be utilized when the positive pre-ictal prediction was made by the physiological model without very high confidence. The confidence may be determined based on the model's outputted prediction probabilities, which are values that represent the likelihood of a class. Values closer to 1 indicate a very high prediction probability of an upcoming seizure, whereas values closer to 0 indicate a very high probability of not experiencing a seizure. After the physiological model provides an alert, the patient may optionally perform an audio test; if the audio test also predicts an upcoming seizure, then a secondary alert is presented to the user, which acts as a “second opinion”. The audio “second opinion” may not be recommended when the confidence score is very high (e.g., between 0.9 and 1.0). The audio “second opinion” may also not recommended when the confidence score is very low (e.g., between 0 and 0.5) in which case the system will not have a seizure alert. The audio “second opinion” may only be recommended when the system provides a seizure alert with a confidence score between 0.5 inclusive and 0.9 inclusive.

The methodology of the data preprocessing and model building for the audio/dysarthria model are detailed below.

For the audio-based prediction, a simple Recurrent Neural Network (RNN) may be implemented and trained on normal and dysarthria (pre-ictal) data. All modeling may be performed using the Keras TensorFlow library. The model may include one LSTM layer, one SimpleRNN layer, and one LSTM layer, each of which was followed by a dropout layer and utilized ReLU activation. The model may also include a dense layer that utilized ReLU activation and an output layer that utilized Sigmoid activation. The model may be validated using a random selection of 20% of the training data. The model may use the Adam optimizer with a learning rate of 0.001. The model may be trained for 10 epochs and tested with the test dataset.

The mobile application may further include a user interface to present the alerts and aid the user in the personalization process.

The mobile application may include a processor that predicted upcoming seizures using the deep learning model(s) and that also trained the model(s) using the patient's data. The wearable device 300 may be configured to continuously (every 10 seconds) send the patient physiological data to the mobile application via Bluetooth. The mobile application may be configured to continuously listen to incoming physiological data, and the mobile application may be configured to perform predictions every 10 seconds using the previous 30-second data. When the prediction results confirmed an upcoming seizure, the mobile application may be configured to display a seizure alert on the mobile application, and the seizure alert was also displayed on the wearable device 300. The processor may interchangeably referred to as controller.

Additionally, the mobile application may also include a data storage that receives and stores physiological data. The patient data may be saved for at least 24 hours on the mobile device 400 before it is deleted. This saved data was used for on-device prediction and used for on-device training to perform the personalization of the model. The approach of on-device training and testing was utilized rather than training and testing the model on a cloud for several reasons: 1) eliminate the need to connect to a different system for predictions and training, 2) offer patient data privacy by keeping the patient's data on patient's phone, 3) offer better scale to support millions of DRE patients by having each patient's model on the patient's phone/mobile device 400.

The patient or caregiver may install the mobile application on his/her mobile phone and may start using the system. The wearable device, worn by the patient, continuously (e.g., every 10 seconds) may be configured to send the patient's physiological data to the mobile application (e.g., via Bluetooth or WiFi). The mobile application may be configured to store the patient's physiological data in its data storage.

The processor of the mobile application may combine the most recently received 10-second segment of data with previously received and stored 20-second segment of data to form a 30-second segment of data. The processor of the mobile application may test the 30-second segment data against the on-device physiological model, which predicts whether a seizure is upcoming. That is, the mobile application may perform predictions using the previous 30-second segment of received data. These 10, 20 and 30-second references are provide an example scenarios only. The system can be configured to different timings as well.

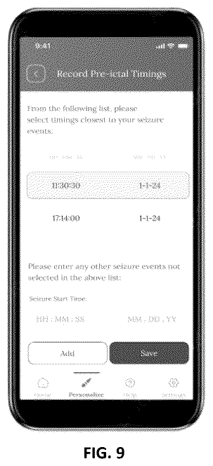

Based on the physiological model's prediction (e.g., when the prediction results confirm an upcoming seizure), the mobile application may be configured to alert the patient/caregiver about the patient's state through the mobile application's user interface. As shown in FIG. 8, the mobile application may be configured to display a seizure alert on the mobile app of the patient/caregiver when the prediction results confirm an upcoming seizure. The mobile application may also be configured to send the patient's state or prediction results to the wearable device 300 so the seizure alert will also be displayed on the wearable device 300.

When the positive pre-ictal prediction was made by the physiological model without very high confidence, the system may recommend the patient to optionally provide a voice or video recording. The system may be configured to display this recommendation to the patient/caregiver on the user interface of the mobile application and the patient then records a voice or video clip using the user interface on the phone/mobile device 400. The processor of the mobile application may be configured to test the voice or video clip from the patient against the on-device audio/dysarthria model or on-device video model and provide a second or a third opinion.

A system for seizure forecasting is provided. The system comprises a wearable device, and a mobile device. The wearable device includes one or more sensors that are configured to obtain physiological data of a user that describes pre-ictal, ictal and post-ictal and normal phases of the user's seizure related activity. The mobile device includes at least a processor, one or more machine learning models and a memory. The memory is encoded with instructions that, when executed by the at least processor of the system, cause the system to perform operations comprises periodically receiving the physiological data of the user from the wearable device; and analyzing the received physiological data using the machine learning models and forecast upcoming seizures. In one embodiment, the forecasting may include predicting the upcoming seizures. In one embodiment, the forecasting may include predicting or forecasting the seizure activity of the user in the pre-ictal phase. In one embodiment, the forecasting may include predicting or forecasting the seizure activity of the user anytime before the ictal phase. In one embodiment, the upcoming seizures may include seizure that may happen in future.

The memory encoded with instructions that, when executed by the at least processor of the system, cause the system to perform operations further comprises: for the seizures detected, prompting user to confirm the identified pre-ictal timings so data for the confirmed timings is used to further train the machine learning model(s). The memory encoded with instructions that, when executed by the at least processor of the system, cause the system to perform operations further comprises: for the seizures detected, allowing the users to explicitly enter seizure start times and end times data so the data for the corresponding pre-ictal timings is used to further train the machine learning model(s).

A method for seizure forecasting may include one or more procedures. For example, the method may include a first procedure in which at least five physiological parameters/metrics of a patient/user are measured or sensed continuously by the one or more sensors 600 of the wearable device 300 to obtain sensor data/information. During this procedure, the wearable device 300 may also be configured to transmit the sensor data to a mobile application (e.g., on a mobile phone/device 400 of the user) continuously. The mobile application may be configured to use AI trained models (in the mobile application and/or in the cloud application) to identify an upcoming seizure based on the received sensor data. The mobile application and/or wearable device 300 may also be configured to provide a preliminary signal/alert to the patient when an upcoming seizure is detected.

The alert signal may be sent to the patient via the wearable device 300 of the patient and/or via the mobile device 400 (on which the mobile application is residing) of the patient. The alert signal may include an audio signal, a vibration signal, or a visual signal (including a vibration, sound, light, etc.) that notifies the patient about an upcoming seizure. The alert signal may optionally be sent to any other personnel configured in the mobile application (including a family member, a caregiver, or a doctor).

In one exemplary scenario, the alert signal may prompt the patient to speak specific words or specific sentences (as in a second procedure described in detail below) and/or to position the mobile device 400 in front of the patient's face and then to speak specific words or specific sentences (as in a third procedure described in detail below). The mobile application may then use all the information for more accurate prediction or forecasting of an upcoming seizure.

The method may include a second procedure in which the patient may be prompted/asked by the application (e.g., on a mobile device 400 of the user) to speak specific words or specific sentences. During this procedure, the application is configured to receive and record the user's responses (words/sentences of the user). The application may be configured to use AI trained models (in the application) to identify if there is any abnormality or voice pattern related to an upcoming seizure.

The method may include a third procedure in which the patient may be prompted/asked by the application (e.g., on a mobile device 400 of the user) to position the mobile device 400 in front of the patient's face and may then be prompted/asked by the application (e.g., on a mobile device 400 of the user) to speak specific words or specific sentences. During this procedure, the application is configured to receive and record the user's responses (words/sentences of the user). The application may be configured to use AI trained models (in the application) to identify if there is any abnormality or visual patterns related to an upcoming seizure.

In addition to or alternatively, during the third procedure, instead of prompting for specific words or specific sentences, the application may prompt the patient to position the mobile device 400 in front of the patient's face and then perform some easy to follow along displayed neurological tests.

The first, the second and third procedures described above may be performed in any sequence. The first, the second and third procedures described above may be performed simultaneously. The first, the second and third procedures described above may be performed independently of each other. That is, each of the first, the second and third procedures may be performed independently to forecast the seizures. The first, the second and third procedures described above may be performed in any combinations thereof. For example, (a) the first and the second procedures, (b) the first and the third procedures, (c) the second and the third procedures, or (d) the first, the second and the third procedures may be performed to forecast the seizures. When multiple procedures are performed, there may be more than one alert provided to the user. For example, there may be an alert after analyzing sensor data and, after analyzing and confirming the user voice data, there may be another confirmation alert.

The system of the present patent application may include multiple AI models 1300′/1300″. The AI models may interchangeably referred to as machine learning models. The multiple AI models may include sensor data AI model, voice pattern AI model, and visual pattern AI model. The sensor data AI model may interchangeably referred to as physiological AI (or deep learning) model. The voice pattern AI model may interchangeably referred to as audio/dysarthria AI (or deep learning) model. Further, the sensor data AI model may include one or more senor AI models each of which may be configured to process/handle data from a sensor. In order to forecast seizures, the AI models may leverage different algorithms including but not limited to Long Short-Term Memory (LSTM), Regression, Recurrent Neural Networks (RNN), Convolutional Neural Networks (CNN) etc. Other algorithms may be used for forecasting, for processing voice data, for processing sequences of data, for processing video data etc. The sensor data AI model may be configured to receive and process the sensor data, and to determine and classify/forecast whether the received sensor data is related to an upcoming seizure. The voice pattern AI model may be configured to receive/obtain and process the voice pattern data, and to determine and classify/forecast whether the received voice pattern data is related to an upcoming seizure. The visual pattern AI model may be configured to receive/obtain and process the visual pattern data, and to determine and classify/forecast whether the received visual pattern data is related to an upcoming seizure. The AI models may be used together or may be used independently of each other or in any combination of models.

The system of the present patent application may include the mobile application. The system may include a cloud application and the mobile application. The cloud and the mobile applications may communicate with each other and in real time. The cloud application may reside on a cloud device/server 500, while the mobile application may reside on a mobile device 400. Any/all of the AI models may run in the mobile application. All of the sensor data AI model, the voice pattern AI model, and the visual pattern AI model may reside on the mobile application. In one embodiment, the sensor data AI model and the voice pattern AI model may reside on the mobile application, while the visual pattern AI model may reside on the cloud application. In another embodiment, the sensor data AI model may reside on the mobile application, while the voice pattern AI model and the visual pattern AI model may reside on the cloud application. Any/all/none of the AI models may run in the cloud application. Any/all of the AI models may be in the cloud application.

In one exemplary scenario, the wearable device 300 may transmit sensor data to the mobile application. In one embodiment, the sensor data may be sent continuously. In one embodiment, the sensor data may be sent periodically. The mobile application may also be configured to obtain the voice pattern data of the patient (using the microphone) and/or obtain the visual pattern data of the patient (using the camera and the microphone). The mobile application may include three AI models (sensor data AI model, voice pattern AI model, and visual pattern AI model). The mobile application may process the received sensor data, the obtained voice pattern data, and the obtained visual pattern data using the AI models on the mobile application. The mobile application may process predictions made by the three AI models and provide an alert to the patient (via mobile application or wearable device 300).

In another exemplary scenario, the wearable device may transmit sensor data to the mobile application. The mobile application may also be configured to obtain the voice pattern data of the patient (using the microphone) and obtain the visual pattern data of the patient (using the camera and the microphone). The mobile application may include two AI models (the sensor data AI model, and the voice pattern AI model), while the visual pattern AI model may be saved in the cloud application. The mobile application may process the received sensor data and the obtained voice pattern data using the AI models on the mobile application. The mobile application may be configured to transmit and receive data/information to/from the cloud application. The transmission of the data/information between (a) the mobile application and the cloud application, (b) the mobile application and the wearable device, and (c) the cloud application and the wearable device may be carried out wirelessly. The mobile application may be configured to process and transmit the visual pattern data from the mobile application to the cloud application. The visual pattern AI model in the cloud application may process the received visual pattern data and determine/classify/forecast whether the received visual pattern data is related to an upcoming seizure. This determination may be transmitted back from the cloud application to the mobile application. The mobile application may further process predictions made by all the AI models and provide an alert to the patient (via mobile application or wearable device 300).

Yet another exemplary scenario is similar to the one described above, except that the mobile application may include only one AI model (the sensor data AI model), while the voice pattern AI model and the visual pattern AI model may be in the cloud application. The mobile application may be configured to process and transmit the voice pattern data and the visual pattern data from the mobile application to the cloud application. The voice pattern AI model in the cloud application may process the received voice pattern data and determine/classify/forecast whether the received voice pattern data is related to an upcoming seizure. The visual pattern AI model in the cloud application may process the received visual pattern data and determine/classify/forecast whether the received visual pattern data is related to an upcoming seizure. These determinations of the visual pattern AI model and the voice pattern AI model may be transmitted back from the cloud application to the mobile application. The mobile application may further process predictions made by all the AI models and provide an alert to the patient (via mobile application or wearable device 300).

Although the patent application describes one AI model for each sensor data, one AI for visual pattern data, and one AI model for audio pattern data, in another embodiment, the patent application may include one AI model configured to process the sensor data, visual pattern data and the audio pattern data. The models may use different machine learning algorithms including LSTM, RNNs, CNNs, Random Forest etc., based on the fitness and the efficacy of the algorithm for the respective model. For example, LSTM classification may be used for the sensor data, and RNNs may be used for the voice data.

Each of the AI models may be trained on a training dataset. For example, the sensor data AI model may be trained on the sensor data training set, the voice pattern AI model may be trained on the voice pattern data, and the visual pattern AI model may be trained on the visual pattern data. Another example, there may be multiple sensor data AI models trained with the corresponding sensor data. Although the AI models are discussed in the patent as three separate models, in one embodiment, the system may include only a single model trained with different data including sensor data, voice pattern data and/or visual pattern data. In another embodiment, the system may include more than one model trained with combinations of data.

Each of the AI models may be trained or pre-trained. For example, the sensor AI model may be pre-trained using five or more sensor data obtained from multiple patients during (1) the patient's normal (not pre-ictal, not ictal, and not post-ictal) activity/phase, (2) the patient's pre-ictal activity/phase, (3) the patient's ictal activity/phase, and (4) the patient's post-ictal activity/phase. The sensor data AI model may be trained on sequences of sensor data (e.g., 30 sec intervals) covering all (normal, pre-ictal, ictal, and post-ictal) phases. The voice pattern AI model may be trained using voice pattern data obtained from multiple patients during (1) the patient's normal (no pre-ictal, no ictal, no post-ictal) activity/phase, (2) the patient's pre-ictal activity/phase, (3) the patient's ictal activity/phase, and (4) the patient's post-ictal activity/phase. The voice patterns may be different in each of the activities/phases, so training the voice data representing each of these activities/phases enables the AI model to learn and recognize/predict voice patterns related to the corresponding activities/phases for new/test data. The visual pattern AI model may be trained using visual pattern data obtained from multiple patients during (1) the patient's normal (no pre-ictal, no ictal, no post-ictal) activity/phase, (2) the patient's pre-ictal activity/phase, (3) the patient's ictal activity/phase, and (4) the patient's post-ictal activity/phase. Training the visual data representing each of these activities/phases enables the AI model to learn and recognize/predict visual patterns related to the corresponding activities/phases for new/test data. Each of the trained AI models may be configured to be updated with more/additional/subsequent data.

Even though this patent application primarily discussed about specific sensor data, audio data and visual data (pictures and video), other physiological data obtained from non-invasive means (e.g., EEG data) and some invasive means (e.g., implanted sensor) may be used for training the models and predicting/forecasting the seizures by the system.

The forecasting/prediction of the seizure may provide a confidence score for the occurrence of the seizure. For example, the confidence score for the forecast of the seizure may be 75% (or 0.75). If the system is configured with multiple AI models, confidence score may be provided for the forecast/prediction for each AI model, so an overall confidence score may be calculated based on the confidence scores of the individual AI models. Each individual AI model may itself be assigned with a weightage, so that an AI model with more weightage may influence the overall confidence score more (e.g., higher score). For example, if the voice data AI model is configured with more weightage, then the confidence score (based on the observed voice patterns) of the voice data AI model may have more influence on the overall confidence score of the seizure forecast.

A confidence score threshold may be configured in the system, so that a seizure alert may be provided to the user if the overall confidence score exceeds the confidence score threshold. For example, if the confidence score threshold is set with 60% (0.60), the alert will be provided only if the overall confidence score provided by the AI models is above 60% (0.60). In addition, the alert may be different depending on the overall confidence score. For example, a higher overall confidence score may provide a more urgent alert (e.g., louder sound).

When data used to train the AI models comprises data corresponding to multiple patients, then the AI models are referred to as general AI models. The general AI models of the present patent application may be configured to further undergo personalized training. That is, each of the general AI models in the mobile application or the cloud application may be trained using the data that is specific/primarily corresponds to a patient/user. In other words, each of the AI models may first be trained using the data from different/multiple patients (excluding the specific patient/user) as described in detail above to create general AI models and then the general AI models may be trained/updated using the patient specific data to generate personalized AI models for the specific patient. Given sufficient training data that corresponds to the patient/user, a personalized AI model may give more accurate results when used with the specific patient/user for whom it was personalized for, compared to using general AI models. Updating the AI models using patient specific data may be done continuously, periodically (hourly, daily, weekly, monthly, quarterly, every minute, every second/sub second etc.) when there is no seizure event detected and/or when there is a (detected or recorded) seizure event. In another embodiment, the AI model may be trained only with the seizure data from the specific patient/user, so it can be used/forecasted for that user.

The general AI models may be trained with different data from patients exhibiting different types of epilepsy (e.g., focal, tonic-clonic). In addition to the general AI models, the application (mobile or cloud) may also have multiple specific epilepsy type AI models. Each specific epilepsy type AI model may be trained using data from patients having a specific type of epilepsy. That is, the patients having a specific type of epilepsy may be grouped together as a specific epilepsy type patient group and the data from that specific epilepsy type patient group may be used to train the corresponding specific epilepsy type AI model. For example, the specific epilepsy types may include but are not limited to (a) focal onset seizures (that start in, and affect, just one part of the brain); (b) generalized onset seizures (that affect both sides of the brain at once and happen without warning. The person will be unconscious (except in myoclonic seizures), even if just for a few seconds and afterwards will not remember what happened during the seizure); (c) unknown onset seizures (that are sometimes used to describe a seizure if doctors are not sure where in the brain the seizure starts. This may happen if the person was asleep, alone or the seizure was not witnessed); and (d) clonic seizures (that involve repeated rhythmical jerking movements of one side or part of the body or both sides (the whole body) depending on where the seizure starts). The specific epilepsy types may also include seizures that may be described depending on a person's level of awareness during their seizures; this means whether or not they are aware of the seizure and what is happening around them. These seizures may include focal aware seizures or focal impaired awareness seizures. The application (mobile/cloud) may be configured to determine whether a specific patient falls under one of the epilepsy type patient groups based on the patient input data and to use the corresponding epilepsy type specific AI model based on that determination. The determination may be done based on a profile similarity between the specific patient and the patient data of the epilepsy type patient group with which the epilepsy type AI model is trained. The application may use a machine learning (AI) algorithm or other algorithm to adaptively learn how to classify the specific patient into one of the epilepsy type patient groups. The user of the mobile application may have an option in the mobile application to personalize with specific epilepsy type AI models rather than the general AI models.

In one exemplary scenario, a new patient/user may initially use a pre-trained general AI model(s) and/or an epilepsy type specific AI model(s). However, as the new patient/user continues to use the system, it is possible to train the model(s) (personalize) more and more with the specific training samples (that correspond to the user) and, thus, generate personalized AI models for that (new) user.

In one scenario, the mobile application may be configured to receive and process the data (e.g., automatically and/or continuously) from the wearable device of the patient, and to determine when a seizure event occurs for that specific patient. That is, the seizure events of the patient are automatically detected by the mobile application and the seizure event related data (including pre-ictal phase, ictal phase, post-ictal phase) are automatically recorded by the mobile application. The mobile application may then be configured to train the AI models using the detected and recorded seizure event data specific to the patient to provide personalized AI models for the patient. In addition to the data from pre-ictal phase, ictal phase and post-ictal phase, the mobile application may be configured to continuously train on data from the normal phase as well.

In another scenario, the mobile application may be configured to receive and process the data (e.g., automatically and/or continuously) from the wearable device of the patient, and to determine when a seizure event occurs for that specific patient. That is, the seizure events of the patient are automatically detected by the mobile application. The mobile application may also be configured to then prompt the user to confirm the data (including pre-ictal data) related to the detected seizure. For example, the mobile application may be configured to display the detected seizure data (including the time of the seizure, the duration of the seizure, other seizure related data) to the user on a user interactive display of the mobile application/device and/or wearable device and may prompt the user to confirm the detected seizure related data via the user interactive display. The confirmed seizure event related data may be recorded by the mobile application. The mobile application may then be configured to train the AI models (present in the mobile application or cloud application) using the detected and user confirmed seizure event data specific to the patient to provide personalized AI models for the patient. The user confirmed seizure data will allow for improved training and improved prediction accuracy of the AI model(s).

In the above two scenarios, the mobile application may be configured to automatically identify (and label) each of the seizure segments as normal, pre-ictal phase (before the seizure begins), as ictal phase (when the seizure occurs), and as post-ictal phase (after the seizure). For example, the seizure duration is recorded as ictal phase, x number of minutes (e.g., 10 min) before the seizure start is recorded as pre-ictal phase, x number of minutes (e.g., 10 min) after the seizure ending is recorded as post-ictal phase.

In yet another scenario, the mobile application may be configured to receive the information related to the seizure from the user. For example, upon user's request, the mobile application may be configured to display various seizure data input options to the user on a user interactive display of the mobile application/device. The user entered seizure event related data (including the time of the seizure, the duration of the seizure, other seizure related information) may be recorded by the mobile application. The mobile application may then be configured to train the AI models using the user received seizure event data specific to the patient and the previously recorded sensor, voice and/or visual data (during the user entered time frame) if exists to provide personalized AI models for the patient. In this exemplary scenario, the user proactively enters the seizure event related data to personalize the AI models. The mobile application may be configured to label the user received seizure event data as an ictal phase (when the seizure occurs) and may be configured to determine the pre-ictal phase (before the seizure begins) and the post-ictal phase (after the seizure) based on the user received seizure event data.

The mobile application may also provide an option to the patient to allow the patient to share his/her seizure event data with the mobile application so this data may be used by the mobile application to continuously train and improve the AI models for all the patients using the mobile application or the epilepsy type specific AI models for a subset of related patients using the mobile application.

FIG. 9 shows an image of the personalization feature in the mobile application. FIG. 9 shows patient-specific personalization in which the patient can enter pre-ictal data/information. The patient can confirm correctly predicted seizure timings after the seizure event. The data from these confirmed timings will be fed into the AI model(s) for on-device personalized training, improving the patient's predictions. The patient-specific personalization in FIG. 9 is effective at addressing the issue of different patients having different pre-ictal patterns.

With personalization, the system predicted with an accuracy in the range between 90% and 95%. Thus, this patent application used patient-specific personalization as an effective way to address the prediction accuracy issue.

The system architecture also considered other key aspects such as patient data privacy, scale of the system to support millions of DRE patients, location agnostic (can be used anywhere in the world), offline capability (work without internet connection), and ease of use, so that the system was designed and implemented to meet these needs expected in the real world usage.

In addition to significantly higher model performances with an optional “second opinion” system, the system also supports several other requirements to make this system practical for real-world usage by 23 million DRE patients. For example, some of the key needs supported by the system are discussed here. In one embodiment, given that the system comprises a wearable device with common sensors and a mobile application, it offers a non-invasive, affordable, and practical solution. In one embodiment, the system can be used anywhere as it works with or without WiFi. In one embodiment, the predictions and training are done on-device, rather than on the cloud, which enables the following: (1) provides patient data privacy by keeping the patient data on the patient's/caregiver's phone and not sending it to the cloud, (2) no need to connect to an external system to make predictions or perform training, making it location-agnostic and providing offline capability, and (3) scales well for millions of patients, as personalization needs one model for each patient and the model resides on the patient's phone. In one embodiment, patients or caregivers themselves can perform personalization using the easy-to-use interface on the mobile without any assistance from the healthcare providers. In one embodiment, the system provides a portable and practical system for 24-hour use.

Although the present patent application has been described in detail for the purpose of illustration, it is to be understood that such detail is solely for that purpose and that the present patent application is not limited to the disclosed embodiments, but, on the contrary, is intended to cover modifications and equivalent arrangements that are within the spirit and scope of the appended claims. In addition, it is to be understood that the present patent application contemplates that, to the extent possible, one or more features of any embodiment can be combined with one or more features of any other embodiment.

The illustration of the embodiments of the present patent application should not be taken as restrictive in any way since a myriad of configurations and methods utilizing the present patent application can be realized from what has been disclosed or revealed in the present patent application. The systems, features and embodiments described in the present patent application should not be considered as limiting in any way. The illustrations are representative of possible embodiments and methods to obtain the desired features.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Terms of degree such as “generally,” “substantially,” “approximately,” and “about” may be used herein when describing the relative positions, sizes, dimensions, or values of various elements, components, regions, layers and/or sections. These terms mean that such relative positions, sizes, dimensions, or values are within the defined range or comparison (e.g., equal or close to equal) with sufficient precision as would be understood by one of ordinary skill in the art in the context of the various elements, components, regions, layers and/or sections being described.

The foregoing illustrated embodiments have been provided to illustrate the structural and functional principles of the present patent application and are not intended to be limiting. To the contrary, the present patent application is intended to encompass all modifications, alterations and substitutions within the spirit and scope of the appended claims.

What is claimed is:

1. A system for seizure forecasting, the system comprising:
a wearable device, the wearable device comprising one or more sensors that are configured to obtain physiological data of a user that describes pre-ictal, ictal and post-ictal and normal phases of the user's seizure related activity; and
a mobile device, the mobile device comprising at least a processor, one or more machine learning models and a memory encoded with instructions that, when executed by the at least processor of the system, cause the system to perform operations comprising:
periodically receiving the physiological data of the user from the wearable device; and
analyzing the received physiological data using the machine learning models and forecast upcoming seizures.

2. The system of claim 1, wherein the memory encoded with instructions that, when executed by the at least processor of the system, cause the system to perform operations further comprises:
for the seizures detected, prompting user to confirm the identified pre-ictal timings so data for the confirmed timings is used to further train the machine learning model(s).

3. The system of claim 1, wherein the memory encoded with instructions that, when executed by the at least processor of the system, cause the system to perform operations further comprises:
for the seizures detected, allowing the users to explicitly enter seizure start times and end times data so the data for the corresponding pre-ictal timings is used to further train the machine learning model(s).
